(19) United States
(12) Patent Application Publication
(10) Pub. No.: US 2004/0086552 A1
(43) Pub. Date: May 6, 2004

(54) Transdermal therapeutic system for highly dispersed silicon dioxide

(76) Inventors: Karin Klokkers, F (DE); Kai-Thomas Kramer, Bad job (DE); Martina Wilhelm, Holzkirchen (DE)

Correspondence Address: Mr. Scott A. McCollister, Fay, Sharpe, Fagan, Minich & McKee LLP, 1100 Superior Ave 7th Floor, Suite 700, Cleveland, OH, 44114-2518, US

(21) Appl. No.: 10/332,864

(22) PCT Filed: Jul. 12, 2001
(86) PCT No.: PCT/EP01/08070

(30) Foreign Application Priority Data
Jul. 12, 2000 (DE) 100 33 853.4

(51) Int. Cl. A61K 9/70

(52) U.S. Cl. 424/449

(57) ABSTRACT

The invention relates to a transdermal therapeutic system comprising a surface layer which is impervious with respect to an active ingredient; a self-adherent matrix layer or a plurality of matrix layers, wherein the exposed matrix layer is, at least self-adherent when the system is applied. Said system also comrises a pull-off protective coating, whereby the matrix layer(s) contains one or more active ingredients and/or one or more biologically active substances and highly dispersed silicon dioxide. Said system contains silicon dioxide in order to increase skin permeation.

The invention relates to highly disperse silica as a penetration promoting substance in transdermal therapeutic systems containing an active agent.

Transdermal administration offers a number of advantages for a number of pharmaceutical agents or other biologically active substances:

The skin is completely available

There is no change in milieu as in oral application

Handling is easy and convenient

Normally a single dose is sufficient instead of repeated daily doses

Patient compliance is much better

Long-term treatment is possible

The release of the medication takes place approximately with a zero order kinetic

Treatment can be discontinued more quickly

A constant plasma level is assured for a long time

Any initially excessively high plasma level as in intravenous administration is avoided and in some cases a lower dose is possible than in oral administration by obviating the initial dose, so that a lower rate of side effects occurs.

The danger of overdosing or underdosing is reduced.

A controlled release of medication is assured especially with a lower therapeutic indication.

In many cases medications which, despite low dosage and high potency, are an ideal choice, have such a low skin permeation that it is impossible to achieve therapeutic plasma levels with simple transdermal therapeutic systems. With all these drugs it is necessary to add so-called permeation promoters to the system in order to increase their absorption into the circulatory system.

Penetration promoting substances must have the following ideal properties in addition to their specific purpose:

Both under occlusive and under nonocclusive conditions they must be compatible with the skin even when left a relatively long time thereon, they must have no allergenic potential and must be compatible with the medication.

The enhancers referred to in the literature can be associated with various chemical classes:

1. Primary and secondary alcohols

1.1 Short-chain primary alcohols C₂ to C₈

1.2 Long-chain primary alcohols C₄ to C₁₆

1.3 Secondary alcohols C₃ to C₂₀

2. Tensides

2.1 Anionic tensides such as Na-dodecylsulfate, for example

2.2 Cationic tensides (e.g., cetylpyridinium chloride) and amines

3. Saturated and unsaturated fatty acids

4. Azones and derivatives (1-alkyl azacycloheptan-2-one, 1-alkyl azacycloalkanone)

5. Amides such as N,N-diethyl-3-methylbenzamide (DEET), N,N-diethyl-m-toluamides

6. Alkyl-N,N-dialkylaminoacetate

7. Macrocyclic ketones and lactones

8. Pyrrolidones

9. Esters such as ethyl acetate, isopropyl myristate, glycerin monolaurate, diethyl sebacate, propylene glycol esters of saturated fatty acids

10. Terpenes such as limonene, menthol, cineol

11. Phospholipids

12. Organic acids such as citric acid, salicylic acid, etc.

The great number of different substances of various chemical structure with known penetration promoting action makes a single mechanism of action appear improbable. So then various mechanisms and combinations of mechanisms will also be discussed.

1. Solvent effects with respect to medication and skin lipids

2. Effects on the three-dimensional lipid structure of the membrane

3. effects on keratin and the protein structure of the skin

Due to the great number of interactions within the skin and the differing chemical nature of the medication, it is difficult to predict the penetration promoting properties of all these enhancers with respect to an active agent.

Experience shows that it is very rare that a penetration promoting substance or a particular mixture of several active agents or groups thereof provide the required properties.

The problem to which the invention is addressed is the preparation of a penetration promoting substance which is compatible with the skin and the active agent, and has no allergenic potential. Furthermore, it should be easily accessible and economical and at the same time have a penetration promoting action on more than one active agent.

Surprisingly it has now been found that highly disperse silica has the property of substantially increasing the penetration of active agents and/or other biologically active substances through the skin.

Highly dispersed silica, also known under the name Aerosil® of the firm of Degussa-Hüls, is commonly used for thickening, thixotropication and strengthening. Aerosil brings about a considerable improvement of the mechanical properties of all elastomers, such as tensile strength, rip propagation or tear resistance. In liquids containing solids, e.g., pigmented varnishes, Aerosil prevents or retards settling. The agglomeration and caking of solid particles of substances in powder form is prevented by Aerosil as a flow adjuvant, so that packing, storage and handling as assured even in high humidity and pressure conditions.

FR 5381 describes Aerosil as a thickener for topical ointments. DE 3416248 describes the addition of colloidal silica to a plaster matrix to improve viscosity. FR 2547502 describes a matrix for transdermal therapeutic systems in which colloidal silica is added as a thixotropic agent.

JP 2512565 B2 (Database Chemical Abstracts, AN 115:263477)

JP 2503095 B2 (Database Chemical Abstracts, AN 116:262553)

JP 06065066 (Database Chemical Abstracts, AN 121:42787) and

JP 04368323 (Database Chemical Abstracts, AN 118:175811) describe plasters in which Aerosil® used astatin.

JP 09169636 Chemical Abstracts, AN 127:86139) describes silica in a plaster to reduce skin irritation.

Transdermal therapeutic systems are also to be found in the following documents: DE 198 27 732, DE 44 05 898, DE 43 09 830, DE 42 30 588, WO 92/20 339; WO 91/05 529, U.S. Pat. No. 5,939,090 and U.S. Pat. No. 5,676,968. This state of the art, however, gives no suggestion of adjusting skin permeation with silica. The problem is solved according to the invention by highly disperse silica which acts as a penetration promoting substance in transdermal therapeutic systems containing active agents or other biologically active substances.

Aerosil®90, Aerosil®130, Aerosil®150, Aerosil®200, Aerosil®300, Aerosil®380, Aerosil®OX50, Aerosil®TT600, Aerosil®MOX80, Aerosil®COK84, Aerosil®R202, Aerosil®R805, Aerosil®R812, Aerosil®812S, Aerosil®R972, and/or Aerosil® R974 or any other highly disperse silica, especially Aerosil®200 and/or Aerosil®R972 can be used according to the invention as highly disperse silica.

The transdermal therapeutic system which contains the permeation promoter silica according to the invention is a plaster. This plaster can be a matrix or membrane system which has an impermeable cover layer and a removable protective layer. In a membrane system the reservoir or reservoir layer lies between the cover layer and the membrane.

An impermeable cover layer can consist of films of acetal, acrylate, acrylonitrile-butadiene-styrene, acrylonitrile (methyl methacrylate) copolymer, acrylonitrile copolymer, ethylene ethyl acrylate, ethylene methyl acrylate, ethylene vinyl acetate, ethylene vinyl acetate copolymer, ethylene vinylalcohol polymer, ionomers, nylon (polyamide), nylon (polyamide) copolymer, polybutylene, polycarbonate, polyester, polyethylene terephthalate, thermoplastic polyester copolymer, polyethylene copolymer (high density), polyethylene (high-molecular-weight, high-density), polyethylene (intermediate-molecular-weight, high-density), polyethylene (linear low density), polyethylene (low density), polyethylene (medium density), polyethylene oxide, polyimide, polypropylene, polypropylene (coated), polypropylene (oriented) polypropylene polystyrene, polyurethane, polyvinyl acetate, polyvinyl chloride, polyvinylidene chloride and/or styrene-acrylonitrile, which if necessary can be metallized or pigmented.

The removable protective layer can be made of polyester, polyethylene, polypropylene, polysiloxane, polyacrylate, ethylene vinyl acetate, polyurethane, polyisobutene or paper, usually coated with silicone and/or polyethylene, or a mixture thereof.

A matrix plaster consists of a cover layer impermeable to the active agent, of one or more self-adhesive matrix layer or layers containing the active agent, or one or more matrix layers which are coated with a contact adhesive, and a removable protective layer.

The medically common matrix formers, such as polyacrylate, silicone, polyisobutylene, rubber, rubber-like synthetic homo-, co- or block polymers, butyl rubber, styrene/isoprene copolymer, polyurethanes, copolymers of ethylene, polysiloxanes, styrene/butadiene copolymer or a mixture thereof, as they are provided in the state of the art. The adhesives can be polydimethylsiloxane, polyacrylates, polyisobutylene, polyacrylate with alkylalcohol esters with 4 to 10 carbon atoms, amine-resistant silicone in ethylacetate or n-heptane, polyisobutylene/mineral oil or a mixture thereof.

Another embodiment of the invention is a membrane system. This consists of a cover layer impermeable to the active agent, a reservoir or reservoir layer containing active substance, a semipermeable membrane, a contact adhesive layer, and a removable protective layer. The matrix (the matrices) or the reservoir contains the active agent or agents and in some cases stabilizers, emulsifiers, thickeners, permeation promoters and/or common matrix adjuvants, membrane system adjuvants or reservoir plaster adjuvants. If necessary, cellulose derivatives, such as, e.g., methylcellulose, hydroxy-propyl cellulose, hydroxyethylcellulose or carboxymethylcellulose, and/or carboxyvinyl polymer, polyacrylates, sodium-plyoxilate or a mixture thereof can be used as gel formers.

The membrane, which usually consists of inert polymers, especially those based on polyethylene, polypropylene, polyvinylacetate, polyamide, ethylene-vinyl-acetate copolymers and/or silicone, can have a controlling action on the release of the active agent on the basis of their pore size.

A pressure-sensitive adhesive based, for example, on polyurethanes, polyisobutylenes, polyvinyl ethers, silicones, polyacrylate or a mixture of these, can be selected as a pressure-sensitive adhesive coating.

The silicone-based adhesive may be a pressure-sensitive silicone adhesive, which is based on two main components, namely a polymer or adhesive, especially polysiloxane and a resin, which increases the adhesiveness. The polysiloxane adhesive usually is prepared with a cross-linking agent for the adhesive, typically with a high molecular weight polydiorganosiloxane, and with the resin, in order to form a three-dimensional silicate structure with an appropriate organic solvent. The admixture of the resin to the polymer is the most important factor for changing the physical properties of the polysiloxane adhesive (see, for example, Sobieski, et al., “Silicone Pressure Sensitive Adhesives”, Handbook of Pressure Sensitive Adhesive Technology, 2^{nd} ed, pp, 508-517 (D. Satas, ed.), Van Nostrand Reinhold, New York, (1989)

Trimethylated silica, which has been treated with polydimethylsiloxane with terminal trimethylsiloxy groups, is a further example of a pressure-sensitive adhesive.

In the case of the adhesives based on polyacrylates, they can be any homopolymer, copolymer or terpolymer, consisting of various acrylic acid derivatives.

Thus, the polyacrylates can be polymers of one or more monomers of acrylic acid and other copolymerizable monomers. Moreover, the acrylate polymers can include copolymers of alkyl acrylates and/or methacrylates and/or copolymerizable secondary monomers or monomers with functional groups. If the amount of any kind that is added as monomer is varied, the cohesive properties of the resulting acrylate polymer are changed. In general the acrylate polymer consists of at least 50 wt. % of an acrylate, methacrylate, alkylacrylate or alkylmethacrylate monomer, 0 to 20% of a functional monomer copolymerizable with acrylate, and 0 to 50% of another monomer.

The following is a list of examples of various acrylate monomers, such as acrylic acid, methacrylic acid, butylacrylate, butylmethacrylate, hexylacrylate, hexylmethacrylate, isooctylacrylate, isooctylmethacrylate, glycidylmethacrylate, 2-hydroxyethylacrylate, methylacrylate, methyl methacrylate, 2-ethylhexylacrylate, 2-ethyl hexylmethacrylate, decylacrylate, decylmethacrylate, dodecylacrylate, dodecylmethacrylate, tridecylacrylate and tridecylmethacrylate, which can be polymerized alone or in mixture.

In addition, functional monomers can be used for the copolymerization which are copolymerizable with the above-named acrylates, such as acrylic acid, methacrylic acid, maleic acid, maleic anhydride, hydroxyethylacrylate, hydroxypropylacrylate, acrylamide, dimethylacrylamide, acrylonitrile, dimethylaminoethylacrylate, dimethylaminoethylmethacrylate tert.-butylaminoethylacrylate, tert.-butylaminoethylmethacrylate, methoxyethylacrylate, vinyl acetate and methoxyethylmethacrylate.

Additional details and examples of pressure-sensitive acrylates which are suitable for the invention are described in Satas Handbook of Pressure Sensitive Adhesive Technology “Acrylic Adhesives”, 2^{nd} ed., pp. 396-456 (D. Satas, ed.), VanNostrand Reinhold, New York (1989).

The penetration promoting highly disperse silica according to the invention is incorporated in the adhesive layer. It is uniformly distributed in the latter. To achieve a uniform distribution the silica must generally swell with the adhesive upon incorporation. During the swelling phase the adhesive and silica mixture must be mixed for a long time with a suitable apparatus, e.g., an Ultraturax.

The content of highly dispersed silica with respect to the matrix weight of the adhesive layer of the transdermal therapeutic system can amount to 0.1-10 wt. %, especially 2-5 wt. % and preferably 2, 4 or 5 wt. %.

The active agent contained in the transdermal therapeutic system can be a representative, for example, from the group of the androgens, estrogens, gestagens, proton pumping inhibitor, 5-HT, antagonists, sympatholytica, sympathomimetica, anticholinergica, tranquilizers and anxiolytica, antiaddictives, analgesics, calcium antagonists, antiemetics, vasodilators, anticoagulants, anti-Parkinsonism agents, antidementia drugs/cholinesterase inhibitors, ACE inhibitors, antihistaminics, ulcer therapeutics/H₂-receptor blockers, angiotensin-II-antagonists, neuroleptics, antidepressives, local anesthetics and/or lipid lowering agents.

The transdermal therapeutic system can contain one or more representatives of the group of the androgenics, e.g., testosterone, testosterone undecanoate, androsterone and/or their derivatives and/or their pharmaceutically unobjectionable salts as active agent components.

The transdermal therapeutic system can contain as active components one or more representatives of the group of the estrogens, e.g., estradiol, estradiol benzoate, estradiol valerate, estradiol dipropionate, ethinylestradiol and/or their derivatives and/or their other pharmaceutically unobjectionable salts.

The transdermal therapeutic system can contain as active components one or more representatives of the group of the gestagens, e.g., progesterone, cyproterone acetate, cyproterone, chlormadinone, chlormadinone acetate, medroxyprogesterone acetate, levonorgestrel, norgestrel, norgestimate, norethiestrone acetate and/or their derivatives and/or their other pharmaceutically unobjectionable salts.

The transdermal therapeutic system can contain as active components one or more representatives of the group of the proton pumping inhibitors, e.g., omeprazole, esomeprazole, lansoprazole, leminoprazole, pantoprazole, rabeprazole, polaprezinc and/or their derivatives and/or their pharmaceutically unobjectionable salts.

The transdermal therapeutic system can contain as active components one or more representatives of the group of the migraine treatment agents and 5-HT₁-antagonists, e.g., lisuride, sumatriptan, sumatriptanhydrogen succinate, rizatriptan, rizatriptan benzoate, almotriptan, avitriptan, eletriptan, frovatriptan, Naratriptan, zolmitriptan and/or their derivatives and/or their their other pharmaceutically unobjectionable salts.

The transdermal therapeutic system can contain as active components one or more representatives of the group of the sympatholytic and sympathomimetic agents, e.g., adimolol, albuterol, alpenolol, amosulalol, arotinolol, atenolol, bambuterol, betaxolol, bevantolol, bisoprolol, bitolterol, bopindolol, broxaterol, bucindolol, bucumolol, bufuralol, bunitrolol, bupranolol, butofilolol, carazolol, carbuterol, carteolol, carvedilol, cetamolol, cicloprolol, clenbuterol, cloranolol, crateolol, dihydroargotamine, dihidroergotamine tartrate, dihydroergotamine mesylate, dilevalol, doxazosin, etilefrin, epanolol, esatenolol, fenoterol, formolterol, ibuterol, isoprenalin, labetalol, landiolol, levobetaxolol, levosalbutamol, mabuterol, mepindolol, metipranolol, metoprolol, morazon, nebivolol, nipradilol, norfenefrin, noradrenalin, oxprenolol, picumeterol, pimolol, pindolol, pirbuterol, phenmetrazin, phenylephetric, phentolamine, phenoxybenzamine, prazosin, procaterol, propanolol, rimiterol, reproterol, salbutamol, salmeterol, sulfonterol, terazosin, terbutalin, tertatolol, tienoxolol, tilisolol, timolol, tolazolin, toliprolol, tolubuterol, tamsulosin, clonidin, moxonidin and/or their derivatives and/or their pharmaceutically unobjectionable salts.

The transdermal therapeutic system can contain as active components one or more representatives of the group of the anticholinergics, e.g., oxitropium, atropine, scopolamine base, atropine methyl bromide, atropine methyl nitrate, scopolamine hydrobromide, scopolamine hydrochloride, scopolamine hydroiodide, tropicamide, oxobutinin and/or their derivatives and/or their other pharmaceutically unobjectionable salts.

The transdermal therapeutic system can contain as active agent components one or more representatives of the group of the tranquilizer/anxiolytics, e.g., alprazolam, bentazepam, bromazepam, camazepam, clorazepate, clonazepam, diazepam, etiracetam, etiolam, fludiazepam, flunitrazepam, flutazolam, flutoprazepam, halazepam, ketazolam, loprazolam, lorazepam, lormetazepam, medazepam, metaclazapam, mexazolam, midazolam, nitrazepam, nordazepam, oxazolam, prazepam, temazepam, triazolam and/or their derivatives and/or their pharmaceutically unobjectionable salts.

The transdermal therapeutic system can contain as active components one or more representatives from the group of the antiaddictives, e.g., nicotine, methadone, disulfiram, lobelin and/or their derivatives and/or their pharmaceutically unobjectionable salts.

The transdermal therapeutic system can contain one or more representatives from the group of the analgesics, e.g., alminoprofen, bermoprofen, carprofen, fenoprofen, flobufen, flunoxaprofen, loxoprofen, pelobiprofen, pranoprofen, pentazocin, tilnoprofen, Ximoprofen, zaltroprofen, diclofenac, amfenac, bromfenac, clidanac, etodolac, felbinac, fentiazac, mofezolac, oxindanac, tifurac, indomethacin, piroxicam, ampiroxicam, meloxicam, isoxicam, lornoxicam, tenoxicam, butorphanol, buprenorphine, morphine, hydromorphone, dihydrocodeine, piritramide, levomethadone, fentanyl, amfenac sodium, bromfenac sodium, clidanac sodium, etodolac sodium, felbinac sodium, fentiazac sodium, mofezolac sodium, oxindanec sodium, tifurac sodium, indomethacin sodium, buprenorphine hydrochloride, morphine acetate, hydromorphone hydrochloride, oxycodone hydrochloride, piritramide hydrogen tartrate, levomethadone hydrochloride, fentanyl dihydrogen citrate and/or their derivatives and/or their other pharmaceutically unobjectionable salts as active components.

The transdermal therapeutic system can contain one or more representatives of the group of the calcium antagonists, e.g., amlodipine, arandipine, azelmidipine, bamidipine, benidipine, cilnidipine, efonidipine, felodipine, flordipine, iganidipine, isradipine, lacidipine, lercanidipine, manidipine, nilvadipine, nisoldipine, nitrendipine, palonidipine, pranidipine, vatanidipine, clentiazem and/or their derivatives and/or their pharmaceutically unobjectional salts as active components.

The transdermal therapeutic system can contain one or more representatives from the group of the antiemetics, e.g., azasetron, batanoprid, cleboprid, dazoprid, dolasetron, domperidon, granisetron, itasetron, levosulpirid, nabilon, ondansetron, pancoprid, ramosetron, tropisetron, zatosetron and/or their derivatives and/or pharmaceutically unobjectionable salts as active components.

The transdermal therapeutic system can contain one or more representatives of the group of the vasodilators, e.g., glycerin trinitrate (nitroglycerin), molsidomin and/or their derivatives and/or their other pharmaceutically unobjectionable salts as active components.

The transdermal therapeutic system can contain one or more representatives of the group of the anticoagulants, e.g., certoparin, dalteparin, danaparoid, enoxaparin, nedroparin, reviparin, tinzaparin and/or their derivatives and/or their pharmaceutically unobjectionable salts as active components.

The transdermal therapeutic system can contain one or more representatives of the group of the antiparkinsonism agents, e.g., aptiganel, biperiden, budipin, cabergolin, etacapon, idazoxan, lazabernid, milacermid, moregilin, pergolid (pergolidmesylat, pergolidhydrochloride) pramipexol, quineloran, rasagelin, remacemide, ropinorol, selegilin, talipexol, tolcapon and/or their derivatives and/or their other pharmaceutically unobjectionable salts as active components.

The transdermal therapeutic system can contain one or more representatives of the group of the antidementia agents/cholinesterase inhibitors, e.g., rivastigmin, pyridostigmin, donepezil, tacrin and/or their derivatives and/or their other pharmaceutically unobjectionable salts as active components.

The transdermal therapeutic system can contain one or more representatives of the group of the ACE inhibitors, e.g., alaceprin, benazepril, ceronapril, cilazapril, denapril, fosinopril, imidapril, moexipril, moveltipril, perindopril, quinapril, ramipril, ramiprilat, rentiapril, spirepril, temocapril, trandolapril, utibapril, zofenopril and their esters and/or their derivatives and/or their other pharmaceutically unobjectionable salts as active components.

The transdermal therapeutic system can contain one or more representatives of the group of the antihistaminics, e.g., acrivastin, carebastin, cetirizin, clenbutaerol, descarbethoxyloratadin, dimetinden, ebastin, epinastin, levocabastin, mequitazin, mizolastin, nafamostat, norastemizol, olopatidin, oxatomid rupatadin, tazifyllin, temelastin, traxanox and/or their derivatives and/or their other pharmaceutically unobjectionable salts as active components.

The transdermal therapeutic system can contain one or more representatives of the group of the ulcerotherapeutics/H₂-receptor blockers, e.g., dalcotidin, famotidin, lafutidin, niperdidin, nizatridin, osutidin, pibutidin, pirenzepin, ramixotidin, misoprostol and/or their derivatives and/or their other pharmaceutically unobjectionable salts as active components.

The transdermal therapeutic system can contain one or more representatives of the group of the angiotensin-II-antagonists, e.g., candesartan, candesartan-cilexetil, losartan, tasosartan and/or their derivatives and/or their other pharmaceutically unobjectionable salts as active components.

The transdermal therapeutic system can contain one or more representatives of the group of the neuroleptics, e.g., benperidol, haloperidol, clozapin, flupentixol, fluphenazin, droperidol, melperon, flupentixoldecanoate, fluspirilen, bromperidol, pimozid, trifluprometazin, risperidon, sertindol, trifluoperidol, olanzapin and/or their derivatives and/or their other pharmaceutically unobjectionable salts as active components.

The transdermal therapeutic system can contain one or more representatives of the group of the antidepressives, e.g., citalopram, reboxetin, alprazolam, fluoxetin and/or their derivatives and/or their other pharmaceutically unobjectionable salts as active components.

The transdermal therapeutic system can contain one or more representatives of the group of the local anesthetic drugs, e.g., lidocaine, benzocaine, procaine, tetracaine, bupivacaine, cinchocaine, etidocaine, mepivacaine, butanilicaine, levobupivacaine, ropivacaine and/or their derivatives and/or their other pharmaceutically unobjectionable salts as active components.

The transdermal therapeutic system can contain one or more representatives of the group of the lipid lowering drugs, e.g., simvastatin, atorvastatin, pravastatin, cerivastatin, dalvastatin, itavastatin lovastatin, dextrothyroxime sodium and/or their derivatives and/or their other pharmaceutically unobjectionable salts as active components.

The active substance contained in the transdermal therapeutic system can also, however, be leflunomide, indapamide, hydroxytamoxifen, finasterid, tirofiban, rosiglitazone, poglitazone, montelukast and/or their derivatives and/or their other pharmaceutically unobjectionable salts.

“Pharmaceutically unobjectionable salts of the named basic active substances” is to be understood to mean acid addition salts. These are obtained by the reation of the active substance in its free base form with pharmaceutically unobjectionable acids. Pharmaceutically unobjectionable acids are inorganic acids (e.g., hydrochloric acid, hydrobromic acid, sulfuric acid, nitric acid, phosphoric acid) or organic acids (e.g., acetic, propionic, hydroxy acetic acid, lactic acid, pyruvic acid, oxalic acid, maleic, malonic, succinic, fumaric, malic, tartaric, citric, methanesulfonic, ethanesulfonic, benzenesulfonic, p-toluenesulfonic, cyclohexanesulfaminic, salicylic, p-aminosalicylic and pamoaic acid). Solvates with the active substance are also designated as acid addition salts. Such solvates are, e.g., hydrates, alcoholates and the like. Possible pharmaceutically unobjectionable salts of the said acid active substances are chiefly alkali metal salts and/or alkali metal salts as well as the ammonium salt, such as the potassium, sodium, lithium, calcium, magnesium and ammonium salt.

Active substances, their derivatives and/or their pharmaceutically unobjectionable salts that are easily soluble in water are used as active components in the transdermal therapeutic system.

In addition to the active agent, the reservoir and the matrix layer can contain other known penetration aids known in the state of the art if the penetration of the agent through the skin is not sufficiently high in the absence of the silica of the invention in the transdermal therapeutic system. Monovalent or polyvalent aliphatic, cycloaliphatic and/or aromatic-aliphatic alcohols with up to eight carbon atoms each, e.g., ethanol, 1,2-propanediol, dexpanthenol and/or polyethylene glycol, alcohol and water mixtures, saturated and/or unsaturated fatty alcohols with 8-18 carbon atoms, terpenes, e.g., cineol, carveol, menthone, terpineol, verbenone, menthol, limonene, thymol, cymene, terpinen-4-ol, neomenthol, geraniol, fenchon; mixtures of terpenes and ethanol and/or propylene glycol; tea tree oil; saturated and/or unsaturated cyclic ketones; alkyl-methyl sulfoxides; saturated and/or unsaturated fatty acids with 8-18 carbon atoms, and their esters and salts; natural vitamin E, synthetic vitamin E and/or vitamin E derivatives; sorbitan fatty acid ester and ethoxylated sorbitan fatty acid ester; azone (laurocapram); azone mixed with alcohols; urea; 1-alkylpyrrolidone; block copolymers of polyethylene glycol and dimethyl siloxane with cationic group at one end; isoprpyl myristate, isopropyl palmitate, folate-polyethyleneglycol-liposome, proliposome, polyoxyethylene-10-stearyl ether; mixture of polyoxyethylene-10-stearyl ether and glyceryl dilaurate; dodecyl-2-(N,N-dimethylamino)-propanoltetradecanoate and/or dodecyl-2-(N,N-dimethylamino)-propianate; N-acetylprolinate ester with more than 8 carbon atoms, nonionic tensides, e.g., lauryl ether, esters of polyoxyethylene; ethosome (phospholipid vesicle); dimethyl(arylimino)sulfuran; mixture of oleic acid analogs and propylene glycol; mixture of padimate 0, octylsalicylate, octylmethoxycinnimate, laurocapram; Cetiol® HE, butanol® G or a mixture of individual components can be used as additional permeation aids.

The invention is explained by the following examples, but without limiting the scope of the invention thereby.

### EXAMPLE 1

Comparison of the permeation values of a transdermal therapeutic system (TTS) with and without silica. A polyisobutylene adhesive (MA24A® of AdhesiveResearch USA) was used, and trandolapril as active substance.

Apparatus for skin permeation:

Cells: Modified flow-through cell

Skin: Hairless mouse from female mice

Acceptor medium: 0.9% sodium chloride+0.05% sodium azide, 60 ml per cell

Permeation temperature: 32° C.±0.5° C.

The active agent concentrations are then determined after sampling with HPLC.

| | Permeation (μg/cm²) |
|---|---|
| TTS without silica in the matrix | |
| Trandolapril 10 wt. % | 24 h 4.9-14.4 |
| Eutanol ® G10 wt. % | 48 h 11.9-26.6 |
| MA24A ® 80 wt. % | |
| TTS with silica in the matrix | |
| Trandolapril 10 wt. % | 24 h 37.6-58.0 |
| Eutanol ® G 10 wt. % | 48 h 82.6-118.1 |
| MA24A ® 76 wt. % | |
| Aerosit ® 200 4 wt. % | |

The weight-percentages relate to the matrix weight.

### EXAMPLE 2

Comparison of the permeation of a transdermal therapeutic system (TTS) with and without silica. A polyisobutylene adhesive (MA24A®) and the methanesulfonic acid salt of Trandolapril was used as material, the trandolapril mesylate being formed in situ in the TTS.

Apparatus for the skin permeation:

Cells: modified flow-through cell

Skin: hairless mouse from female mice

Acceptor medium: 0.9% sodium chloride+0.05% sodium azide, 60 ml per cell

Permeation temperature: 32° C.±0.5° C.

| | Permeation [μg/cm²] |
|---|---|
| TTS without silica in the matrix | |
| Trandolapril 10 wt % | 24 h 10.9-13.8 |
| Methanesulfonic acid 2.26 wt % | 48 h 19.2-24.6 |
| Eutanol ® G 5 wt % | |
| MA24A ® 78.7 wt % | |
| TTS with silica in the matrix | |
| Trandolapril 10 wt % | 24 h 36.1-74.4 |
| Methanesulfonic acid 2.26 wt % | 48 h 146.2-204.6 |
| Eutanol ® G 5 wt % | |
| MA24A ® 78.74 wt % | |
| Aerosil ® 200 4 wt-% | |

The weight-percentages refer to the matrix weight.

### EXAMPLE 3

Comparison of the permeation values of a transdermal therapeutic system (TTS) with and without silica. A polyiobutylene adhesive (MA24A®) and ramipril mesylate as the active substance were used.

Apparatus for skin permeation:

Cells: modified flow-through cells

Skin: hairless mouse from female mice

Acceptor medium: 0.9% sodium chloride+0.05% sodium azide, 60 ml per cell.

Permeation temp.: 32 C±0.5 C

| | Permeation [μg/cm²] |
|---|---|
| TTS without silica in the matrix | |
| Ramipril mesylate 15 wt % | 24 h 111-156 |
| Eutanol ® G 10 wt % | 48 h 184-219 |
| MA24A ® 75 wt % | |
| TTS with silica in the matrix | |
| Ramipril mesylate 15 wt % | 24 h 251-338 |
| Eutanol ® G 10 wt % | 48 h 862-997 |
| MA24A ® 70 wt % | |
| Aerosil ® 200 5 wt-% | |

The weight-percentages refer to the matrix weight.

### EXAMPLE 4

Comparison of the permeation of a transdermal therapeutic system (TTS) with and without silica. An acrylate adhesive (Durotak® of National Starch/USA) and ramipril mesylate as active substance were used, the ramipril mesylate being formed in situ.

Apparatus for skin permeation:

Cells: modified flow-through cells

Skin: hairless mouse from female mice

Acceptor medium: 0.9% sodium chloride+0.05% sodium azide, 60 ml per cell.

Permeation temp.: 32 C±0.5 C

| | Permeation [μg/cm²] |
|---|---|
| TTS without silica in the matrix | |
| Ramipril 15 wt % | 24 h 6-29 |
| Methanesulfonic acid 3.8 wt % | 48 h 11-53 |
| Eutanol ® G 10 wt % | |
| Durotak ® 387-2353 71.2 wt % | |
| TTS with silica in the matrix | |
| Ramipril 15 wt % | 24 h 122-248 |
| Methanesulfonic acid 3.5 wt % | 48 h 366-533 |
| Eutanol ® G 10 wt % | |
| Durotak ® 387-2353 66.5 wt.-% | |
| Aerosil ® 200 5 wt-% | |

The weight-percentages refer to the matrix weight.

### EXAMPLE 5

Comparison of the permeation of a transdermal therapeutic system (TTS) with and without silica. An acrylate adhesive (Durotak®)) was used and ramipril mesylate as active agent, the ramipril mesylate being formed in situ.

Apparatus for skin permeation:

Cells: modified flow-through cells

Skin: hairless mouse from female mice

Acceptor medium: 0.9% sodium chloride+0.05% sodium azide, 60 ml per cell.

Permeation temp.: 32 C±0.5 C

| | Permeation [μg/cm²] |
|---|---|
| TTS without silica in the matrix | |
| Ramipril 10 wt % | 24 h 19-24 |
| Methanesulfonic acid 2.4 wt.-% | 48 h 41-48 |
| Eutanol ® G 10 wt % | |
| Durotak ® 387-2510 77.6 wt % | |
| TTS with silica in the matrix | |
| Ramipril 10 wt % | 24 h 51-80 |
| Methanesulfonic acid 2.4 wt % | 48 h 205-253 |
| Eutanol ® G 10 wt % | |
| Durotak ® 387-22510 73.6 wt.-% | |
| Aerosil ® 200 4 wt-% | |

The weight-percentages refer to the matrix weight.

### EXAMPLE 6

Comparison of the permeation of a transdermal therapeutic system (TTS) with and without silica. A polyisobutylene adhesive (MA24A®) was used, and moxonidine base as active agent and oleic acid as enhancer.

Apparatus for skin permeation:

Cells: modified flow-through cell

Skin: hairless mouse from female mice

Acceptor medium: 0.9% sodium chloride+0.05% sodium azide, 60 ml per cell.

Permeation temp.: 32 C±0.5 C

| | Permeation [μg/cm²] |
|---|---|
| TTS without silica in the matrix | |
| Moxonidin 10 wt. % | 24 h 23.0-42.6 |
| Oleic acid 10 wt. % | 48 h 45.3-82.1 |
| MA24A ® 80 WT. % | |
| TTS with silica in the matrix | |
| Moxonidin 10 wt. % | 24 h 58.6-84.8 |
| Oleic acid 10 wt % | 48 h 107.8-159.0 |
| Aerosil ® 200 3 wt % | |
| MA24A ® 77 wt. % | |

The weight-percentages refer to the matrix weight.

### EXAMPLES 7 AND 8

For Ramipril mesilate a skin permeation with Eutanol G should be about 920 μg/cm²/48 h (Example 7) and with Eutanol G/α-tocopherol acetate, about 680 μg/cm²/48 h (Example 8). These specifications are achieved by increasing the content of Aerosil 200; cf. the following table.

| | | Skin permeation |
|---|---|---|
| Charge (Ramipril-TTS) | Composition | [μg/cm²] |
| RAM 0076 TTS | Ramipril Mesilate 15% | 24 h: 294, 202 |
| | Eutanol G 10% | |
| | Aerosil 200 2% | 48 h: 509, 394 |
| | MA 24 73% | |
| RAM 0081 TTS | Ramipril Mesilate 15% | 24 h: 251, 338 |
| | Eutanol G 10% | |
| | Aerosil 200 5% | 48 h 997, 862: |
| | MA 24 70% | |
| RAM 0077 TTS | Ramipril Mesilate 15% | 24 h: 148, 99 |
| | Eutanol G 10% | |
| | α-tocopherol acetate 10% | 48 h: 313, 272 |
| | Aerosil 200 2% | |
| | MA 24 63% | |
| RAM 0083 TTS | Ramipril Mesilate 15% | 24 h: 187, 190 |
| | Eutanol G 10% | |
| | α-tocopherol acetate 10% | 48 h: 653, 708 |
| | Aerosil 200 5% | |
| | MA 24 60% | |

1. Transdermal therapeutic system with a cover coating impermeable to the active substance, with a self-adhesive matrix coating or with a plurality of matrix coatings, of which at least the matrix coat exposed in the application of the system is self-adhesive, and with a removable protective coating, the matrix coating or coatings containing one or more active agents and/or one or more other biologically active substances and highly disperse silica, the system containing silica to increase skin permeation.

2. Transdermal therapeutic system with a cover coating impermeable to the active substance, with one or more matrix coatings with a content of active substance and/or other biologically active matter, the matrix layer or layers being coated with an adhesive (adhesive layer) and with a removable protective layer, the adhesive layer containing a highly disperse silica.

3. Transdermal therapeutic system according to claim 2, characterized in that the system contains silica to increase the skin permeation.

4. Transdermal therapeutic system with a cover coating impermeable to active substance, a reservoir or a reservoir layer with a content of the active substance and/or other biologically active substances, with a semipermeable membrane, with an adhesive coating and with a removable protective layer, the adhesive coating containing highly disperse silica to increase the skin permeation.

5. Transdermal therapeutic system according to any of the foregoing claims, characterized in that the system contains silica in an amount which in a given system (disregarding silica) corresponds to a given permeation value or is adjusted to a given permeation value.

6. Transdermal therapeutic system according to claim 5, characterized by a silica content which corresponds to a maximum permeation value.

7. Transdermal therapeutic system according to any of the foregoing claims, characterized in that the highly disperse silica has been incorporated uniformly into the self-adhesive matrix layer(s) or into the adhesive layer.

8. Transdermal therapeutic system according to claim 7, characterized in that the highly disperse silica has been incorporated by causing a mixture of self-adhesive matrix/silica or a mixture of pressure-sensitive adhesive/silica to swell in the presence of a fluid medium and then forming it into matrix layer(s) or adhesive layer.

9. Transdermal therapeutic system according to any of the foregoing claims, characterized by a content of highly disperse silica of 0.1 to 10 wt-%, especially 2 to 5 wt-% and preferably about 2, about 3, about 4 or about 5 wt-% with respect to the weight of the self-adhesive matrix layer(s) or of the adhesive layer.

10. Transdermal therapeutic system according to any of the foregoing claims, characterized by Aerosil®90, Aerosil®130, Aerosil®150, Aerosil®200, Aerosil®300, Aerosil®380, Aerosil®OX50, Aerosil®TT600, Aerosil®MOX80, Aerosil®COK84, Aerosil®R202, Aerosil®R805, Aerosil®R812, Aerosil®812S, Aerosil®R972 and/or Aerosil®R974 as highly disperse silica.

11. Transdermal therapeutic system according to claim 10, characterized by Aerosil®200 and/or Aerosil®R972 as highly disperse silica.

12. Transdermal therapeutic system according to any of the foregoing claims, characterized by a content of stabilizers, emulsifiers, thickening agents, permeation promoting agents and/or conventional membrane system adjuvants or reservoir plaster adjuvants.
